(19) United States
(12) Patent Application Publication
(10) Pub. No.: US 2025/0072886 A1
(43) Pub. Date: Mar. 6, 2025

(54) SUTURING INSTRUMENT AND SUTURING POSITIONING INSTRUMENT AND SYSTEM

(71) Applicant: Kabushiki Take Wing, Tokyo (JP)

(72) Inventor: Kazunari OKUMA, Tokyo (JP)

(21) Appl. No.: 18/823,292

(22) Filed: Sep. 3, 2024

(30) Foreign Application Priority Data
Sep. 4, 2023 (JP) 2023-142904

(51) Int. Cl. A61B 17/04

(52) U.S. Cl. CPC A61B 17/0469; A61B 17/0485

(57) ABSTRACT

A suturing instrument in which a distal end portion of a collecting member of a straight line shape or a curved shape having a predetermined length of a semi-cylindrical shape or a spatula shape is provided with an opening opened in the intersecting direction with respect to the length direction of the collecting member. A suturing positioning instrument and a system configured to repair a fracture in a meniscus are also disclosed.

### FIELD OF THE INVENTION

The present invention relates to a suturing instrument and a suturing positioning instrument and a system.

### BACKGROUND OF THE INVENTION

Conventionally, there has been widely adopted a suture method in which two needles are provided in a case, the two needles slot in to be inserted into a meniscus, and in this state, a suture thread of a straight line shape is inserted into one of the two needles, a suture thread having a distal end formed in a loop shape is inserted into the other needle, and in a joint capsule, one suture thread of a straight line shape is passed through a loop of the other suture thread (see U.S. Pat. No. 11,013,510 B2).

In the conventional surgical medical instrument, there is the problem that it is very difficult to pass the one straight line suture thread through the loop of the other suture thread in the joint capsule, a suture operation takes time, and a burden on a patient is large.

In addition, since the two needles are slot in to be inserted into the meniscus in a parallel state, there is the problem that a portion where the meniscus can be sutured is limited.

### SUMMARY OF THE INVENTION

The present invention provides a minimally invasive suturing instrument that reduces a burden on a patient.

For this reason, the medical instrument according to the present invention includes a collecting member 1 of a straight line shape or a curved shape having a predetermined length of a semi-cylindrical shape or a spatula shape, and an opening 3 opened in an intersecting direction with respect to a length direction of the collecting member 1 at a distal end portion of the collecting member 1.

### BRIEF DESCRIPTION OF THE DRAWINGS

FIG. 1A is a side view according to an embodiment of the present invention;

FIG. 1B is a side view according to another embodiment;

FIG. 1C is a side view according to another embodiment;

FIG. 1D is a side view according to another embodiment;

FIG. 2A is a longitudinal sectional side view according to the embodiment of the present invention;

FIG. 2B is a plan view of a collecting member according to the embodiment of the present invention;

FIG. 2C is a longitudinal sectional side view according to another embodiment;

FIG. 2D is a plan view of the same according to another embodiment;

FIG. 2E is a longitudinal sectional side view according to another embodiment;

FIG. 2F is a plan view of the same according to another embodiment;

FIG. 3A is a schematic plan view of a state in which a suture thread is inserted into an opening of the collecting member according to the embodiment of the present invention;

FIG. 3B is a schematic plan view of the same according to another embodiment;

FIG. 4A is a schematic plan view of a state in which a second suture thread is inserted into the opening of the collecting member according to the embodiment of the present invention;

FIG. 4B is a schematic plan view of the same according to another embodiment;

FIG. 5A is a side view of a state in which the suture thread is inserted into the opening of the collecting member according to the embodiment of the present invention;

FIG. 5B is a side view of the same according to another embodiment;

FIG. 5C is a side view the same according to another embodiment;

FIG. 6A is a longitudinal sectional side view of a state in which the suture thread is inserted into the opening of the collecting member according to the embodiment of the present invention;

FIG. 6B is a longitudinal sectional side view of the same according to another embodiment;

FIG. 6C is a longitudinal sectional side view of the same according to another embodiment;

FIG. 7A is a longitudinal sectional side view in which the insertion body is brought into contact with a contact portion of the collecting member according to the embodiment;

FIG. 7B is a longitudinal sectional side view in which an insertion body is inserted into the collecting member according to the embodiment;

FIG. 7C is a longitudinal sectional side view of the same according to another embodiment;

FIG. 7D is a longitudinal sectional side view of the same according to another embodiment;

FIG. 7E is a longitudinal sectional side view of the same according to another embodiment;

FIG. 7F is a longitudinal sectional side view of the same according to another embodiment;

FIG. 8A is a longitudinal sectional side view of a state in which the collecting member is inserted into an affected part according to the embodiment of the present invention;

FIG. 8B is a longitudinal sectional side view of the same according to another embodiment;

FIG. 8C is a longitudinal sectional side view of the same according to another embodiment;

FIG. 9A is a state view in which the suture thread is collected by the collecting member according to the embodiment of the present invention;

FIG. 9B is a state view of the same according to another embodiment;

FIG. 10 is a schematic plan view of a state in which the suture thread after connection is drawn;

FIG. 11 is a schematic plan view of a state in which the suture thread after connection is further drawn;

FIG. 12 is a schematic plan view of a state in which the suture thread after connection suture threads the affected part of the meniscus;

FIG. 13A is a schematic plan view of a use state of a suturing handle according to the embodiment;

FIG. 13B is a schematic plan view of a use state of a positioner of the collecting member according to another embodiment;

FIG. 14 is a schematic perspective view of a needle in a state where the needle is inserted into a needle cylinder;

FIG. 15 is a longitudinal front view of the same;

FIG. 16A is a longitudinal exploded view according to the embodiment;

FIG. 16B is a longitudinal assembled view of the suturing handle according to the embodiment;

FIG. 17A is a longitudinal exploded view according to another embodiment;

FIG. 17B is a longitudinal assembled view of the suturing handle according to another embodiment;

FIG. 18A is a front view of a jig that bends the collecting member according to the embodiment;

FIG. 18B is a side view of a jig that bends the collecting member according to another embodiment;

FIG. 18C is a front view of a jig that bends the collecting member according to another embodiment;

FIG. 18D is a use state view of a jig that bends the collecting member according to another embodiment;

FIG. 18E is a use state view of a jig that bends the collecting member according to another embodiment;

FIG. 19 is a schematic view of a state in which the needle is brought into contact with a joint capsule;

FIG. 20 is a schematic view of a state in which the needle and the suture thread are made to pass through the opening of the collecting member;

FIG. 21 is a schematic view of a state in which the needle and the suture thread have passed through the opening of the collecting member;

FIG. 22 is a schematic view of a state in which only the suture thread is indwelled; and

FIG. 23 is a schematic view of a state in which the insertion body is inserted into the collecting member and the suture thread is held.

### DETAILED DESCRIPTION OF THE PREFERRED EMBODIMENTS

An embodiment of the present invention will be described below with reference to the drawings. Reference numeral 1 denotes a collecting member for collecting a suture thread 5 used for suturing an affected part (damaged portion D) of a meniscus H of a joint capsule (inside joint cavity) S. A collecting member 1 is formed to have a straight line shape or a curved shape having a predetermined length of a semi-cylindrical shape or a spatula shape, and is provided with an opening 3 opened in an intersecting direction with respect to a length direction of the collecting member 1 at a distal end portion of the collecting member 1.

Therefore, the collecting member 1 can be easily inserted into the joint capsule having a narrow procedure space, and thus, when the suture thread 5 is inserted from the intersecting direction of the collecting member 1, the suture thread 5 can be inserted into a pair of the openings 3 of the collecting member 1 only by moving the meniscus H straight, and operations of inserting the suture thread 5 into the collecting member 1 and collecting the suture thread 5 from the collecting member 1 can be facilitated.

That is, the meniscus H is located between a femur and a tibia and is usually in contact with each other. At the time of surgery, spaces are secured by manually opening both between the femur and the meniscus H, and between the tibia and the meniscus H, and the collecting member 1 is inserted into the spaces.

When the tissue in the joint is cleaned, a tubular instrument having a diameter of about 3.5 mm is inserted to perform surgery, and the degree of freedom is further increased in the collecting member 1 of a spatula shape, and the ease of suturing is improved.

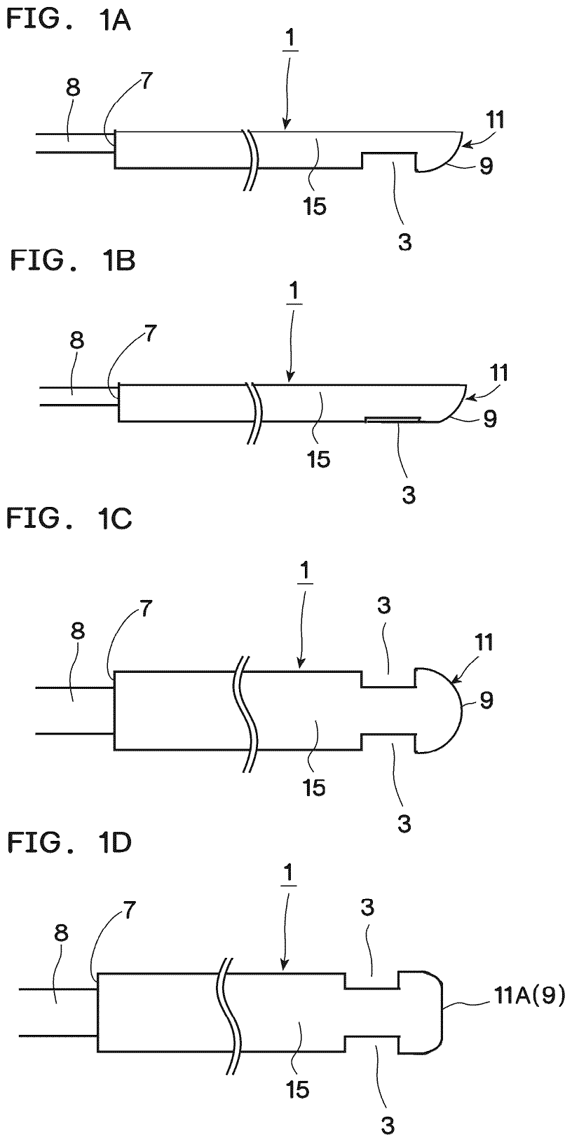

FIG. 1B illustrates another embodiment of the collecting member 1 having a spatula shape, and having a reduced opening area of the opening 3, to improve the rigidity against bending of the collecting member 1.

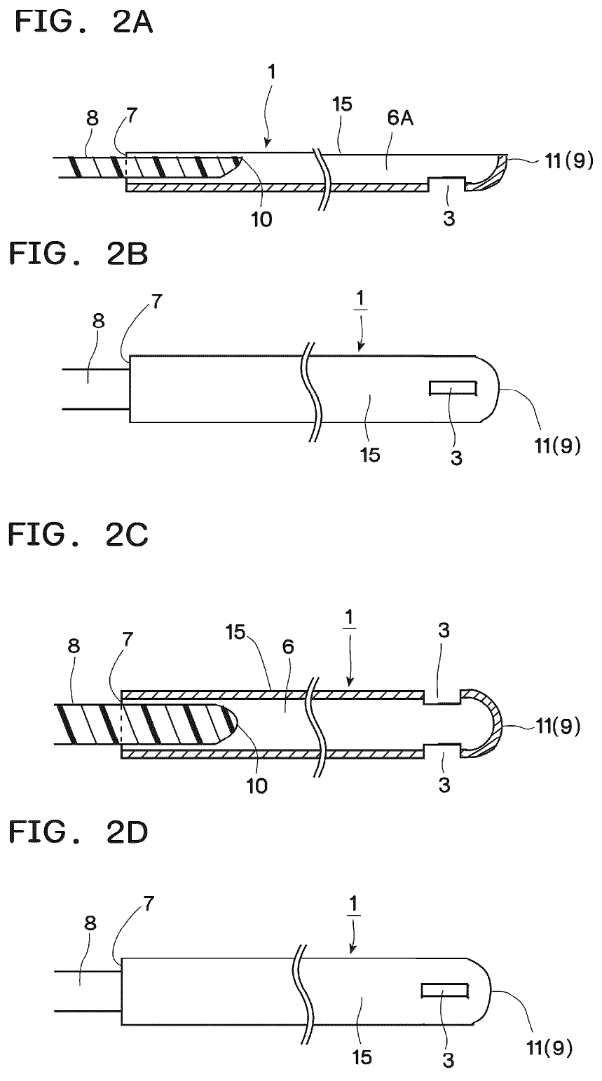

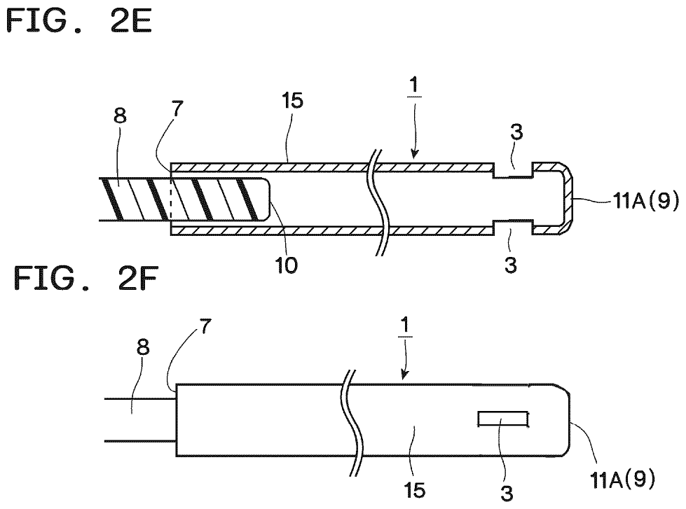

As illustrated in FIG. 2A, the collecting member 1 has a configuration in which a fitting groove 6A is provided on an inner side surface of the collecting member 1, and an insertion body 8 of a semi-axial rod shape is fitted and inserted into a fitting groove 6A from a base side of the collecting member 1.

Therefore, the suture thread 5 inserted into the opening 3 of the collecting member 1 can be pulled out to the outside of a body in a state of being engaged and held by being sandwiched between the outer surface of the insertion body 8 and the inner surface of the collecting member 1, and an operation of ligating the suture thread 5 outside the body can be facilitated.

A tip wall 9 is raised at the distal end of the collecting member 1 (FIG. 2A), and the opening 3 having a predetermined length in the length direction of the collecting member 1 is provided on the collecting member 1 on the base side relative to the tip wall 9.

Therefore, the insertion body 8 fitted and inserted into the collecting member 1 can abut on the tip wall 9 of the collecting member 1 to sandwich the suture thread 5 in the collecting member 1 between the insertion body 8 and the tip wall 9, the operation of pulling out the suture thread 5 to the outside of the body can be facilitated, and the operation of ligating the suture thread 5 outside the body can be facilitated.

The tip wall 9 of the collecting member 1 includes a contact portion 11 on which the distal end portion 10 of the insertion body 8 inserted into the fitting groove 6A from the base side of the collecting member 1 abuts (FIG. 2A).

Therefore, the distal end portion 10 of the insertion body 8 inserted into the collecting member 1 can abut on the contact portion 11 of the collecting member 1, to allow the distal end portion 10 of the insertion body 8 and the contact portion 11 of the collecting member 1 to hold and lock the suture thread 5 in the collecting member 1, and the operation of pulling out the suture thread 5 to the outside of the body can be facilitated, and the operation of ligating the suture thread 5 outside the body can be facilitated.

The distal end portion 10 of the insertion body 8 and the contact portion 11 of the collecting member 1 are configured such that an outer surface of the distal end portion 10 abuts on an inner surface of the contact portion 11 to relatively prevent the suture thread 5 from coming off.

Therefore, the distal end portion 10 of the insertion body 8 fitted and inserted into the collecting member 1 can abut on the contact portion 11 of the collecting member 1, to allow the distal end portion 10 of the insertion body 8 and the inner surface of the contact portion 11 to sandwich the suture thread 5, and the operation of pulling out the suture thread 5 to the outside of the body can be facilitated, and the operation of ligating the suture thread 5 outside the body can be facilitated.

At least the distal end portion 10 of the insertion body 8 is formed with flexibility, and the insertion body 8 is configured to prevent cutting of the suture thread 5 when the distal end portion 10 of the insertion body 8 passes above or near the opening 3 of the collecting member 1.

Therefore, when the distal end portion 10 of the insertion body 8 passes through the opening 3 of the collecting member 1, while the suture thread 5 can be prevented from being cut by an edge of the opening 3, the operation of pulling out the suture thread 5 to the outside of the body can be facilitated, and the operation of ligating the suture thread 5 outside the body can be facilitated.

The opening 3 of the collecting member 1 is formed to have a size that allows at least the suture thread 5 and/or a needle 16 to be inserted into the opening 3.

Therefore, the suture thread 5 can be easily passed through the opening 3 of the collecting member 1, and insertion of the suture thread 5 into the collecting member 1 is facilitated.

A plurality types of the collecting member 1 having different cross-sectional shapes, from a semi-tubular arc shape formed by cutting a tubular member having a perfect circular shape to a spatula shape, having different arc or bending angles, may be prepared and selectively used.

In addition, a rising angle of the tip wall 9 of the collecting member 1 may be any angle as long as the distal end portion 10 of the insertion body 8 can abut on the tip wall 9, and may be any angle as long as the tip wall 9 is laid from 90 degrees (right angle) to about 30 degrees in the horizontal direction.

FIG. 1C illustrates a collecting member 1 according to another embodiment, in which the collecting member 1 is formed in a straight line shape or a curved shape having a predetermined length of a hollow tubular shape, and having a pair of the openings 3 opened in the intersecting direction with an axial direction of the collecting member 1 at the distal end portion of the collecting member 1.

Therefore, the collecting member 1 is inserted in the joint capsule so as to be in a parallel state with respect to a position of an affected part (damaged portion D) of the meniscus H, and in this state, when the suture thread 5 is inserted from the intersecting direction of the collecting member 1, the suture thread 5 can be inserted into the pair of openings 3 of the collecting member 1 only by moving the meniscus H straight, and the operation of inserting the suture thread 5 into the collecting member 1 can be facilitated.

A material of the collecting member 1 is any desired material, but it is preferable to form the collecting member 1 using a tubular member made of metal having a diameter of about 3.5 mm.

An insertion space 6 is provided in the inside of the collecting member 1, and is configured such that an insertion body 8 of an axial rod shape is inserted from a base side insertion port 7 provided on the base side of the collecting member 1. Therefore, the suture thread 5 inserted into the pair of openings 3 of the collecting member 1 can be pulled out to the outside of the body with the suture thread 5 interposed between an outer surface of the insertion body 8 and an inner surface of the collecting member 1, and the operation of ligating the suture thread 5 outside the body can be facilitated. The pair of openings 3 closed at the tip wall 9 of the collecting member 1 and each having a predetermined length in the length direction of the collecting member 1 on the base side of the collecting member 1 relative to the tip wall 9 so as to face each other in the intersecting direction with respect to the length direction of the collecting member 1 (FIGS. 1C and 1D).

Therefore, the insertion body 8 inserted into the collecting member 1 can abut on the tip wall 9 of the collecting member 1 to allow the suture thread 5 in the collecting member 1 to be interposed between the insertion body 8 and the tip wall 9, the operation of pulling out the suture thread 5 to the outside of the body can be facilitated, and the operation of ligating the suture thread 5 outside the body can be facilitated.

The distal end portion 10 of the insertion body 8 and the inner surface of the tip wall 9 of the collecting member 1 are configured such that an outer surface of the distal end portion 10 closely abuts on the inner surface of the tip wall 9 to relatively prevent the suture thread 5 from coming off. Therefore, the distal end portion 10 of the insertion body 8 inserted into the collecting member 1 can abut on the tip wall 9 of the collecting member 1 to allow the suture thread 5 in the collecting member 1 to be interposed between the distal end portion 10 and the inner surface, the operation of pulling out the suture thread 5 to the outside of the body can be facilitated, and the operation of ligating the suture thread 5 outside the body can be facilitated.

That is, a distal end of the collecting member 1 is provided with a contact portion 11 on which the distal end portion 10 of the insertion body 8 abuts, and the distal end portion 10 of the insertion body 8 is brought into contact with the contact portion 11, to allow the suture thread 5 inserted into the collecting member 1 to be interposed and locked between the contact portion 11 and the distal end portion 10.

It is preferable that the tip wall 9 of the collecting member 1 is formed in an arc shape in a side view (FIG. 1C), because damage of the suture thread 5 can be prevented when the suture thread 5 is brought into contact with the contact portion 11.

In other words, the distal end portion 10 of the insertion body 8 and the contact portion 11 of the collecting member 1 are configured such that the outer surface of the distal end portion 10 closely abuts on the inner surface of the contact portion 11 to relatively prevent the suture thread 5 from coming off.

Therefore, the distal end portion 10 of the insertion body 8 inserted into the collecting member 1 can abut on the inner surface of the contact portion 11 of the collecting member 1, to allow the suture thread 5 in the collecting member 1 to be interposed between the distal end portion 10 and the inner surface, the operation of pulling out the suture thread 5 to the outside of the body can be facilitated, and the operation of ligating the suture thread 5 outside the body can be facilitated.

Moreover, at least the distal end portion 10 of the insertion body 8 is formed with flexibility, and the insertion body 8 is configured to prevent cutting of the suture thread 5 when the distal end portion 10 of the insertion body 8 passes above or near the opening 3 of the collecting member 1.

Therefore, when the distal end portion 10 of the insertion body 8 causes the suture thread 5 to be brought into contact with the contact portion 11, an end portion of the opening 3 and the distal end of the insertion body 8 can be prevented from damaging the suture thread 5 like a so-called guillotine.

A portion close to the base side relative to the opening 3 of the collecting member 1 is an insertion portion 15 into which the insertion body 8 is inserted, and an inner diameter of the insertion portion 15 is formed to be larger than an outer diameter of the insertion body 8.

Therefore, insertion body 8 can be inserted into the collecting member 1 easily and smoothly, and the distal end portion 10 of the insertion body 8 can easily abut on the inner surface of the tip wall 9 of the collecting member 1, the operation of pulling out the suture thread 5 to the outside of the body can be facilitated, and the operation of ligating the suture thread 5 outside the body can be facilitated.

The opening 3 of the collecting member 1 is formed to have a size that allows at least the suture thread 5 is inserted into the opening 3, and is configured to have a size that allows the needle 16, to which the suture thread 5 penetrating the meniscus H is attached, to pass through the opening 3.

Therefore, the suture thread 5 together with the needle 16 can be passed through the opening 3 of the collecting member 1, and insertion of the suture thread 5 into the collecting member 1 is facilitated.

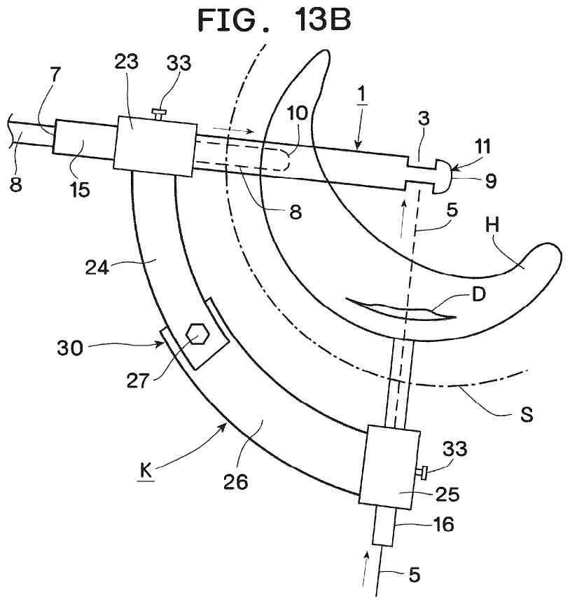

The needle 16 may have any desired configuration as long as the suture thread 5 passes through the needle 16 so that the suture thread 5 can pass through and suture the affected part (damaged portion D) of the meniscus H. In the present embodiment, the needle is formed of a hollow member, and the suture thread 5 is passed through via a hollow needle 16B of 16A to 18G (FIG. 14).

Therefore, the needle 16 may be formed of a single hollow member, but may have a double tubular configuration in which the suture thread 5 is pierced to the hollow needle 16B, and the needle 16B with the suture thread 5 pierced is inserted into the needle 16, to allow the suture thread 5 to pierce the opening 3 of the collecting member 1 (FIG. 14).

In addition, the suture thread 5 is not particularly limited as long as it is generally used for suturing the meniscus H, but it is preferable that the suture thread 5 has the minimum necessary rigidity such that the base side of the suture thread 5 is fed into the needle 16 to allow the distal end of the suture thread 5 to be inserted into the opening 3 of the collecting member 1.

Furthermore, although not illustrated, since it is not necessary to bind the suture thread 5 in the body, a needle may be attached to the distal end of the suture thread 5 to facilitate insertion of the collecting member 1 into the opening 3.

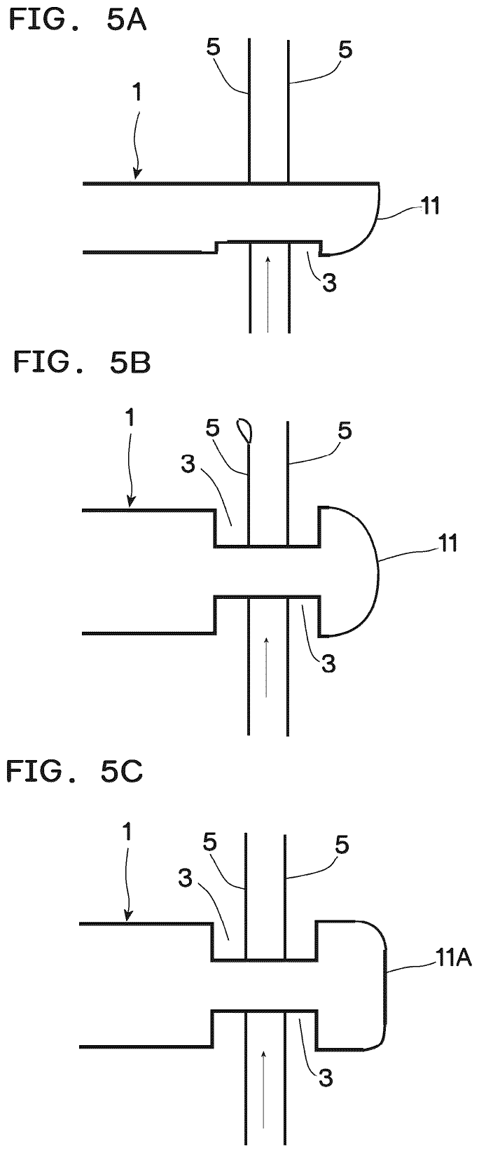

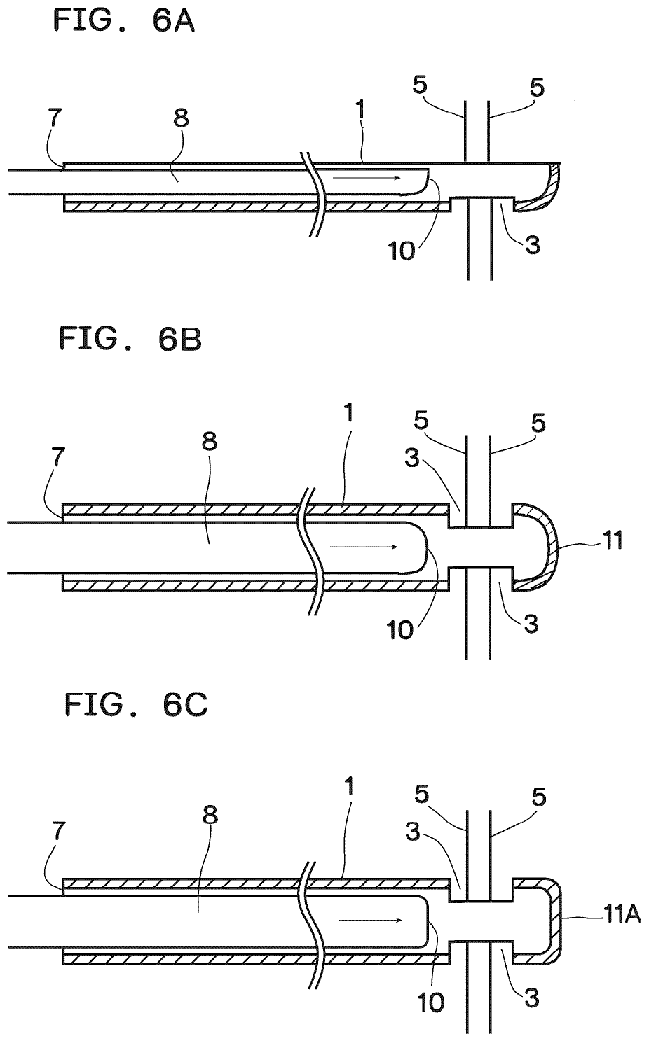

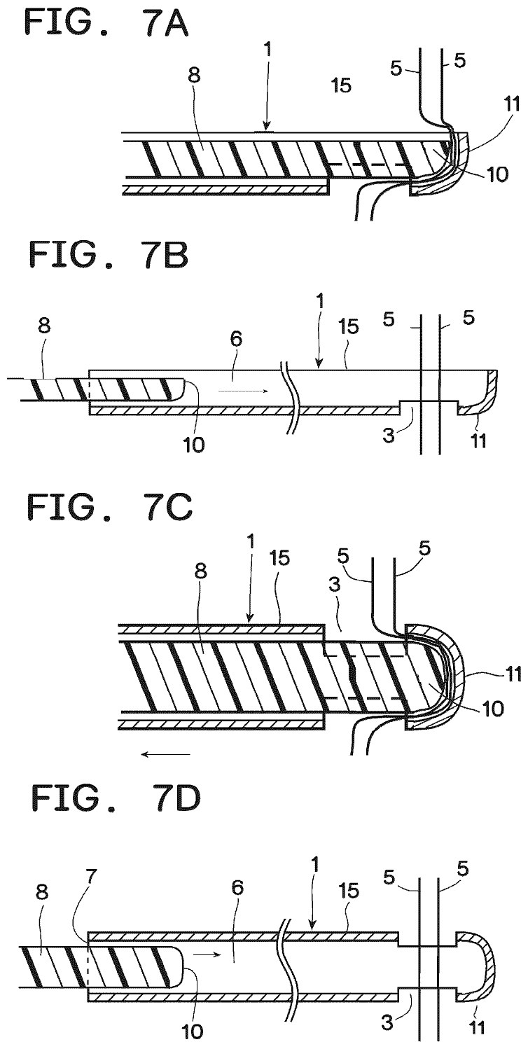

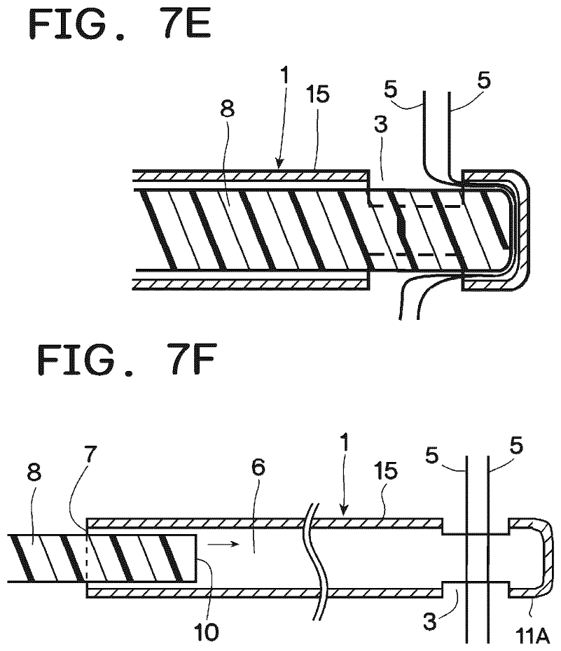

In addition, in the present invention, the collecting member 1 is used to collect the suture thread 5, connect the two suture threads 5 outside the body, return the connected suture thread 5 into the body, and perform the operation of ligating. Therefore, although the distal ends of the two suture threads 5 are both formed in a straight line shape, in order to facilitate the connection of the two suture threads 5 outside the body, the distal end of one of the suture threads 5 may be formed in a loop shape (FIG. 5B).

In addition, the distal end of the collecting member 1 of a straight line shape having a predetermined length, the collecting member 1 being hollow and provided with the insertion space 6 inside, is closed by the tip wall 9, the pair of the openings 3 each having a predetermined length in the length direction of the collecting member 1 are provided in the collecting member 1 on the base side of the collecting member 1 for the predetermined length from the tip wall 9 so as to face each other in the intersecting direction with respect to the length direction of the collecting member 1, the opening 3 is opened to have a size that allows the suture thread 5 or the needle 16 to be inserted to the opening 3, the base side insertion port 7 for inserting the insertion body 8 is formed on the base side of the collecting member 1, and a distal end side of the collecting member 1 beyond the opening 3 is formed in the contact portion 11 on which the distal end portion 10 of the insertion body 8 inserted into the insertion space 6 abuts.

Therefore, the tip wall 9 of the collecting member 1 is inserted from the outside of the body into an approach path formed on a rear side of the meniscus H, the opening 3 of the collecting member 1 is positioned on a back side of the damaged portion (fractured portion) D of the meniscus H, the needle 16 and the suture thread 5 are allowed to enter so as to pass through the damaged portion D of the meniscus H from the approach path on a front side of the meniscus H, one or both of the needle 16 and the suture thread 5 are passed through the opening 3 of the collecting member 1, similarly, another suture thread 5 is passed through the opening 3, the insertion body 8 is inserted into the insertion space 6 of the collecting member 1 in a state of leaving the distal ends of the two suture threads 5 outside the collecting member 1, the distal end portion 10 of the insertion body 8 is brought into contact with the contact portion 11 of the collecting member 1, the collecting member 1 with the two suture threads 5 and their distal ends is pulled out to the outside of the body, and one of the two suture threads 5, after being taken out, of a loop shape is passed through (connected to) the other suture thread 5, and the base of the suture thread 5 of a loop shape is pulled to suture the damaged portion D of the meniscus H with the other suture thread 5.

In this case, although the distal end of one suture thread 5 of the two suture threads 5 has a loop shape and the other suture thread 5 has a straight line shape, this is for facilitating the operation of ligating outside the body, is not a requirement of the present invention, and both distal ends of the two suture threads 5 may have a straight line shape.

It is preferable that the distal end portion 10 of the insertion body 8 is formed in an arc shape in a side view, because damage of the suture thread 5 can be prevented when the suture thread 5 is pushed inside of the opening 3 toward the distal end side.

The collecting member 1 may be bent and formed in an arc shape in a side view. In this case, when the collecting member 1 is formed to have flexibility capable of bending during the operation while having rigidity capable of entering inside the tissue, it is preferable because the operation can be easily performed. In addition, if the flexibility of the collecting member 1 is set to irreversible flexibility, rigidity capable of entering inside the tissue can be secured, which is preferable.

That is, the position of the opening 3 of the collecting member 1 may not match the direction of the meniscus H due to a space problem in the joint capsule, but the opening 3 can be positioned at an appropriate position by bending (curving) the collecting member 1.

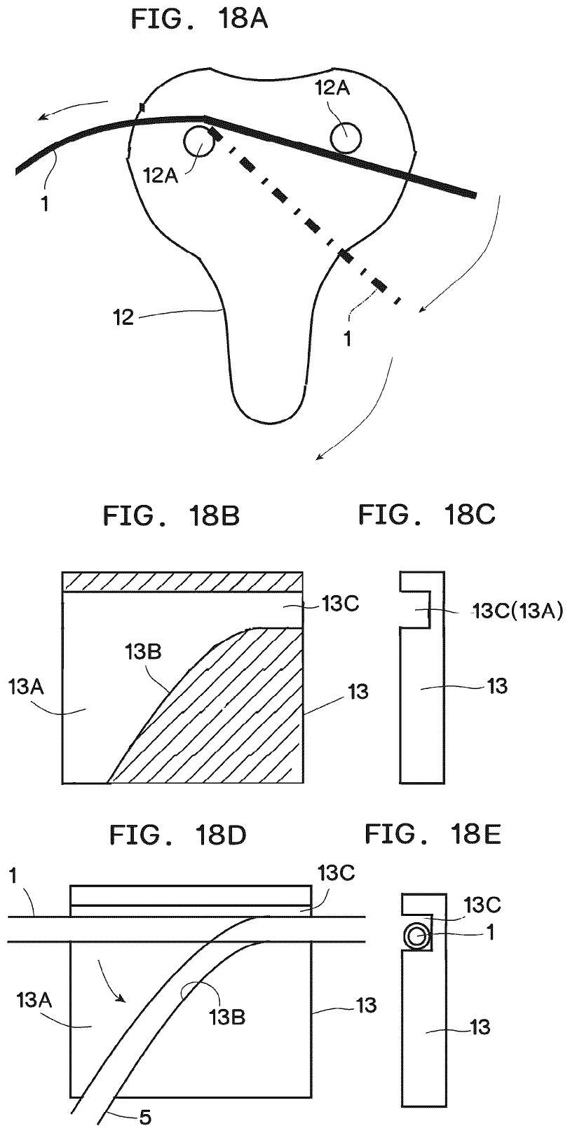

When the collecting member 1 is curved, the collecting member 1 and the needle 16 are adjusted in advance to intersect each other. As illustrated in FIG. 18A, for example, the collecting member 1 is curved by two protrusions 12A of a jig 12.

Although the jig 12 may be used in any manner, it is preferable to use the jig such that the entire collecting member 1 is bent to draw an arc by changing the position of bending little by little, and one part of the collecting member 1 is not intensively bent.

FIG. 18B illustrates a jig 13 according to another embodiment in which the jig 13 has an insertion groove 13A with a curved surface 13B inside the insertion groove 13A, and the distal end of the collecting member 1 inserted into the insertion groove 13A is curved along the curved surface 13B in a state of protruding from an outlet 13C.

FIG. 1C illustrates the collecting member 1 according to another embodiment in which the collecting member 1 has the tip wall 9 having an arc shape. The pair of openings 3 each having a predetermined length in the length direction of the collecting member 1 are provided on the base side of the collecting member 1 relative to the tip wall 9 for the predetermined length from the tip wall 9 so as to face each other in the intersecting direction with respect to the length direction of the collecting member 1.

FIG. 1D illustrates the collecting member 1 according to another embodiment in which the collecting member 1 has the tip wall 9 having an arc shape, and the pair of openings 3 each having a predetermined length in the length direction of the collecting member 1 are provided on the base side of the collecting member 1 relative to the tip wall 9 for the predetermined length from the tip wall 9 so as to face each other in the intersecting direction with respect to the length direction of the collecting member 1, and the distal end side of the collecting member 1 beyond the opening 3 is formed to be a fitting portion 11A to which the distal end portion 10 of the insertion body 8 inserted into the insertion space 6 is fitted.

Therefore, the distal end portion 10 of the insertion body 8 inserted into the collecting member 1 can be fitted to the fitting portion 11A of the collecting member 1, to allow the suture thread 5 in the collecting member 1 to be interposed between a bottom face and a top face of the fitting portion 11A and the inner surface of the tip wall 9, and the operation of pulling out the suture thread 5 to the outside of the body can be facilitated, and the operation of ligating the suture thread 5 outside the body can be facilitated. The distal end portion 10 of the insertion body 8 and the fitting portion 11A of the collecting member 1 are configured such that the outer surface of the distal end portion 10 is closely fitted to the inner surface of the fitting portion 11A to relatively prevent the suture thread 5 positioned inside the fitting portion 11A from coming off.

Therefore, the distal end portion 10 of the insertion body 8 inserted into the collecting member 1 can be fitted to the fitting portion 11A of the collecting member 1, to allow the suture thread 5 in the collecting member 1 to be interposed between the bottom face of the fitting portion 11A and the inner surface of the tip wall 9 and the top face of the fitting portion 11A, and the operation of pulling out the suture thread 5 to the outside of the body can be facilitated, and the operation of ligating the suture thread 5 outside the body can be facilitated. The opening 3 of the collecting member 1 is formed to have a size that allows at least the suture thread 5 to be inserted into the opening 3, and is configured to have a size that allows the suture thread 5 and the needle 16 passing through the meniscus H to pass through the opening 3.

Therefore, the suture thread 5 together with the needle 16 can be passed through the opening 3 of the collecting member 1, and insertion of the suture thread 5 into the collecting member 1 is facilitated.

The distal end of the collecting member 1 of a straight line shape having a predetermined length, the collecting member 1 being hollow and provided with the insertion space 6 inside, is closed by the tip wall 9, the pair of the openings 3 each having a predetermined length in the length direction of the collecting member 1 are provided in the collecting member 1 on the base side of the collecting member 1 for the predetermined length from the tip wall 9 so as to face each other in the intersecting direction with respect to the length direction of the collecting member 1, the opening 3 is opened to a size that allows the suture thread 5 or the needle 16 to be inserted to the opening 3, the base side insertion port 7 for inserting the insertion body 8 is formed on the base side of the collecting member 1, and the distal end side of the collecting member 1 beyond the opening 3 is formed in the fitting portion 11A on which the distal end portion 10 of the insertion body 8 inserted into the insertion space 6 is fitted. (FIGS. 2A to 2C)

Therefore, the fitting portion 11A of the collecting member 1 is inserted from the outside of the body into an approach path formed on a rear side of the meniscus H, the opening 3 of the collecting member 1 is positioned on a back side of the damaged portion (fractured portion) D of the meniscus H, whereas, the needle 16 and the suture thread 5 are allowed to enter so as to pass through the damaged portion D of the meniscus H from the approach path on a front side of the meniscus H, one or both of the needle 16 and the suture thread 5 are passed through the opening 3 of the collecting member 1, similarly, another suture thread 5 is passed through the opening 3, the insertion body 8 is inserted into the insertion space 6 of the collecting member 1 in a state of leaving the distal ends of the two suture threads 5 outside the collecting member 1, the distal end portion 10 of the insertion body 8 is closely fitted to the fitting portion 11A of the collecting member 1, the collecting member 1 together with the distal ends of the two suture threads 5 are pulled out to the outside of the body, and the two suture threads 5 that have been taken out are ligated together, and the base of the suture thread 5 is pulled to suture the damaged portion D of the meniscus H with the two suture threads 5.

That is, when the distal end portion 10 of the insertion body 8 is fitted to the fitting portion 11A, the suture thread 5 is locked in the fitting portion 11A, and the collection of the suture thread 5 is achieved.

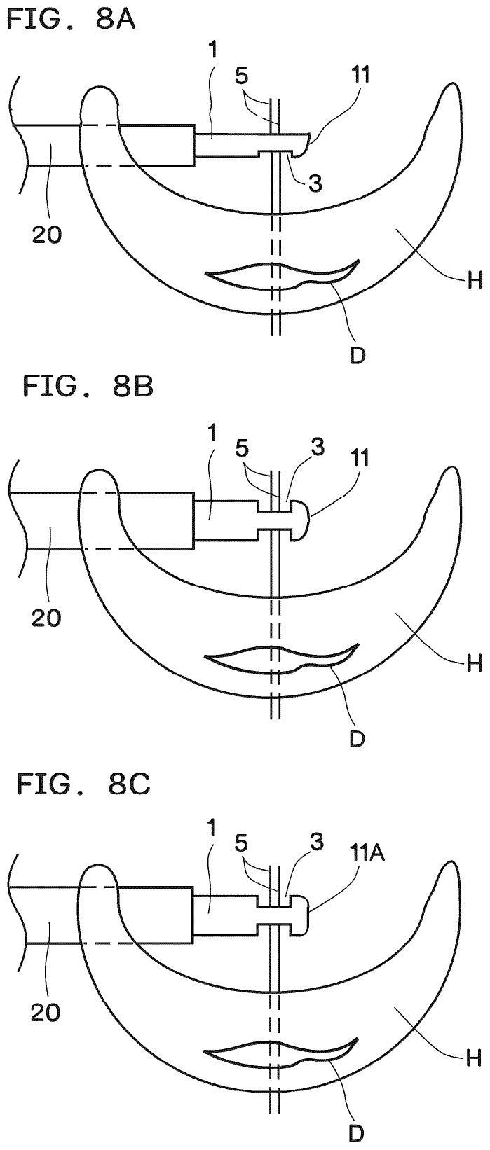

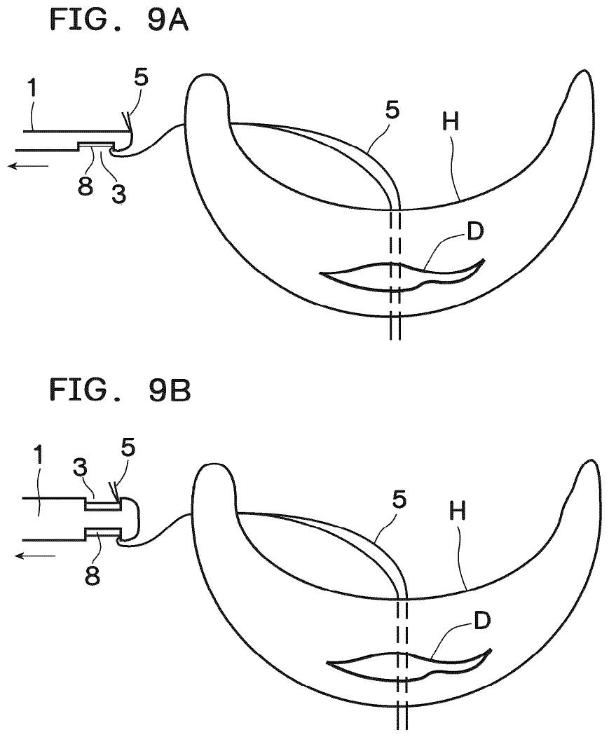

The collecting member 1 is configured to be insertable into a casing 20 having a larger diameter than the outer diameter of the collecting member 1 and having a straight line hollow tubular shape, the casing 20 having been inserted into the back side of the meniscus H (FIG. 8).

Therefore, the casing 20 is inserted in a state of being parallel to the affected part (damaged portion (fractured portion) D) of the meniscus H, the collecting member 1 is inserted into the casing 20, the opening 3 of the collecting member 1 is made to protrude to the distal end side beyond the distal end side of the casing 20, the suture thread 5 or the needle 16 is inserted into the opening 3 of the collecting member 1, the distal end of the suture thread 5 is locked to the distal end of the collecting member 1, the collecting member 1 is taken out of the body while the casing 20 is indwelled in the body in this state, the suture thread 5 is pulled out of the body, and a connection operation with a non-looped suture thread 5 piercing a loop of a looped suture thread 5 can be performed outside the body.

When the collecting member 1 is inserted into the casing 20 again, collecting can be performed with the opening 3 of the collecting member 1 positioned in the vicinity of the meniscus H, and the second suturing can be performed. In this manner, the suture operation of the damaged portion D of the meniscus H can be easily repeated.

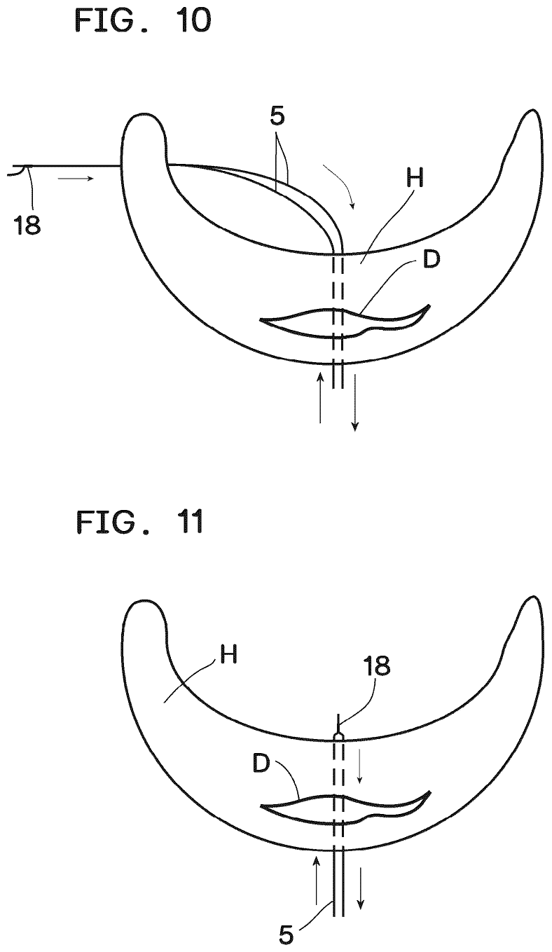

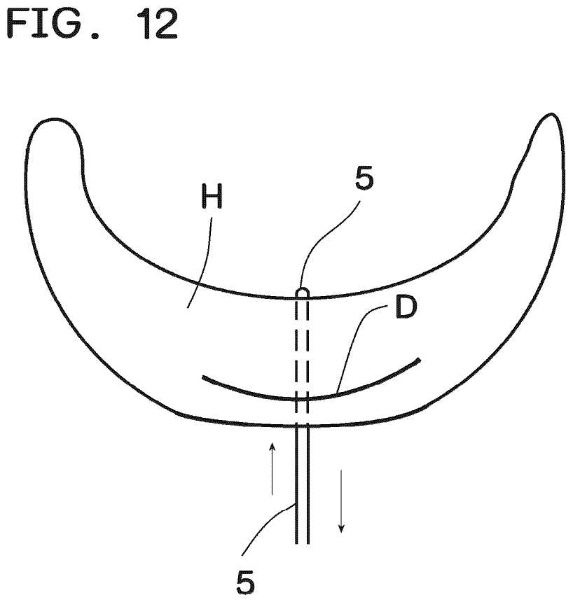

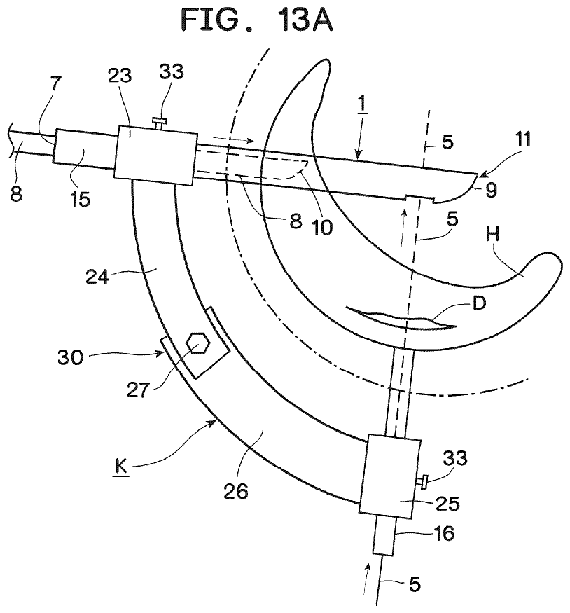

Reference numeral 18 denotes a connecting portion connecting the two suture threads 5 (FIG. 11).

The casing 20 is a member into which the collecting member 1 is inserted. FIG. 8 illustrates a usage example of the collecting member 1 according to an embodiment having a different shape.

The insertion body 8 is formed of a material having flexibility and rigidity such as vinyl and silicon such that the distal end of the insertion body 8 reaches the distal end portion 10 of the collecting member 1 when inserted into the insertion space 6 of the collecting member 1.

The opening 3 of the collecting member 1 is formed in a rectangular slit shape having a width of about 2 mm and a length of 10 to 20 mm.

The positioning instrument K in FIG. 13 includes a handle 30 having one-side handle 24 and other-side handle 26 fixed together with a fixture 27, the one-side handle 24 being provided with an attachment tube 23 to which the collecting member 1 having the opening 3 at the distal end and allowing the suture thread 5 to be inserted is attached, the other-side handle 26 being provided with an attachment tube 25 to which the needle 16 for inserting and guiding the suture thread 5 in the vicinity of the damaged portion D of the meniscus H is attached.

Therefore, the operation of inserting the insertion body 8 into the collecting member 1 can be performed by holding the handle 30, and the operation of inserting the insertion body 8 is facilitated.

In the positioning instrument K, the attachment tube 23 and the attachment tube 25 are arranged each other such that the distal end of the needle 16 is positioned to intersect the opening 3 in a state where the opening 3 of the collecting member 1 is positioned at a predetermined position in the vicinity of the meniscus H.

Therefore, the collecting member 1 and the needle 16 attached to the handle 30 are positioned so as to intersect each other in the body, and the operation of inserting the collecting member 1 and the needle 16 is facilitated.

That is, the handle 30 includes the one-side handle 24 having the attachment tube 23 and the other-side handle 26 movably attached to the one-side handle 24, in which the base of the one-side handle 24 and the base of the other-side handle 26 are overlapped and fixed by a fastener 35.

With this configuration, a length of the handle 30 can be switched and adjusted, and operability and workability can be improved.

Although an attaching configuration of the collecting member 1 to the attachment tube 23 is any desired configuration, as an example, the attachment tube 23 is provided with a fastener 33, and after the collecting member is inserted 1 into the attachment tube 23, the collecting member 1 is fixed by the fastener 33.

The attachment tube 25 for the other side is attached to the base side of the other-side handle 26, and the needle 16 is detachably attached to the attachment tube 25. The attachment tube 25 for the other side is attached to the base side of the other-side handle 26, and the needle 16 is detachably attached to the attachment tube 25.

Therefore, when the distal end of the needle 16 is inserted so as to come into contact with the damaged portion D of the meniscus H and the suture thread 5 is inserted into the needle 16, the suture thread 5 is guided by the needle 16 and can be reliably inserted into the damaged portion D of the meniscus H.

The positioning instrument K allows the attachment tube 23 and the attachment tube 25 of the handle 30 to be arranged to each other such that the distal end of the needle 16 is positioned to intersect the opening 3 in a state where the opening 3 of the collecting member 1 is positioned at a predetermined position in the vicinity of the meniscus H.

Therefore, a predicted position at which the suture thread 5 will be inserted into the damaged portion D of the meniscus H is set in advance, an orientation of the attachment tube 23 of the handle 30 is set such that the opening 3 of the collecting member 1 is positioned at the set position, and then the one-side handle 24 and the other-side handle 26 are combined and fixed such that the attachment tube 25 of the other-side handle 26 is positioned at an angle at which the distal end of the needle 16 will be inserted into a position at which the opening 3 of the collecting member 1 will be positioned.

Therefore, it is possible to simulate an arrangement relationship among the damaged portion D of the meniscus H, the opening 3 of the collecting member 1, and the distal end position of the needle 16 in advance outside the body. Based on this simulation, it is only necessary to insert the collecting member 1 and the needle 16 into the body and insert the suture thread 5 from the needle 16 into the opening 3 of the collecting member 1 and locked thereto. Therefore, the suture operation can be performed in a very short time.

That is, the collecting member 1 and the needle 16 are set so as to cross the opening 3 in a default (initial setting) state. In this case, even when the angle formed by the collecting members 1 and the needle 16 is changed by extending and contracting the one-side handle 24 and the other-side handle 26 with respect to the opening 3, the needle 16 always intersects the opening 3 without requiring special adjustment. Furthermore, in the adjustment of the one-side handle 24 and the other-side handle 26, it is originally desired that the opening 3 and the needle 16 of the collecting member 1 intersect at a right angle. However, the angle may have to be changed due to a space problem in the joint capsule, and the collecting member 1 can be bent for the purpose of matching the direction of the meniscus H.

Therefore, it is possible to simulate an arrangement relationship among the damaged portion D of the meniscus H, the opening 3 of the collecting member 1, and the distal end position of the needle 16 in advance outside the body. Based on this simulation, it is only necessary to insert the collecting member 1 and the needle 16 into the body and insert the suture thread 5 from the needle 16 into the opening 3 of the collecting member 1 and locked thereto. Therefore, the suture operation can be performed in a very short time.

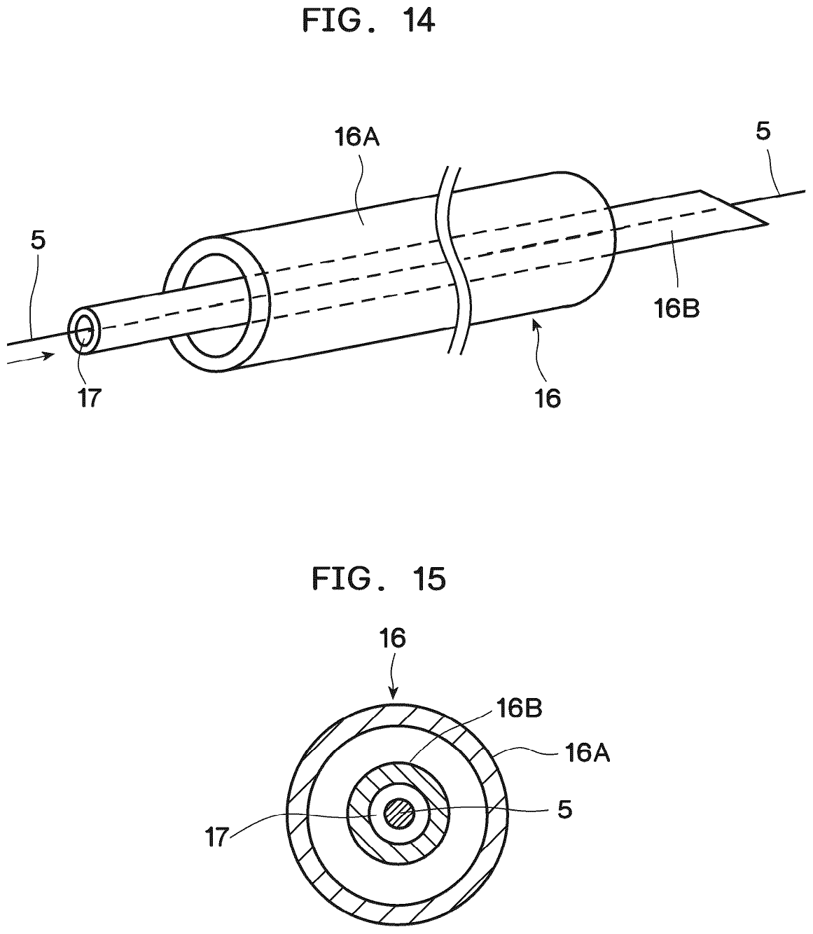

A moving configuration of the one-side handle 24 and the other-side handle 26 of the handle 30 is any desired configuration. As an example, a moving groove 36 is formed in one of the one-side handle 24 and the other-side handle 26, and a moving body 37 that moves by being engaged with the moving groove 36 is provided in the other thereof (FIG. 16).

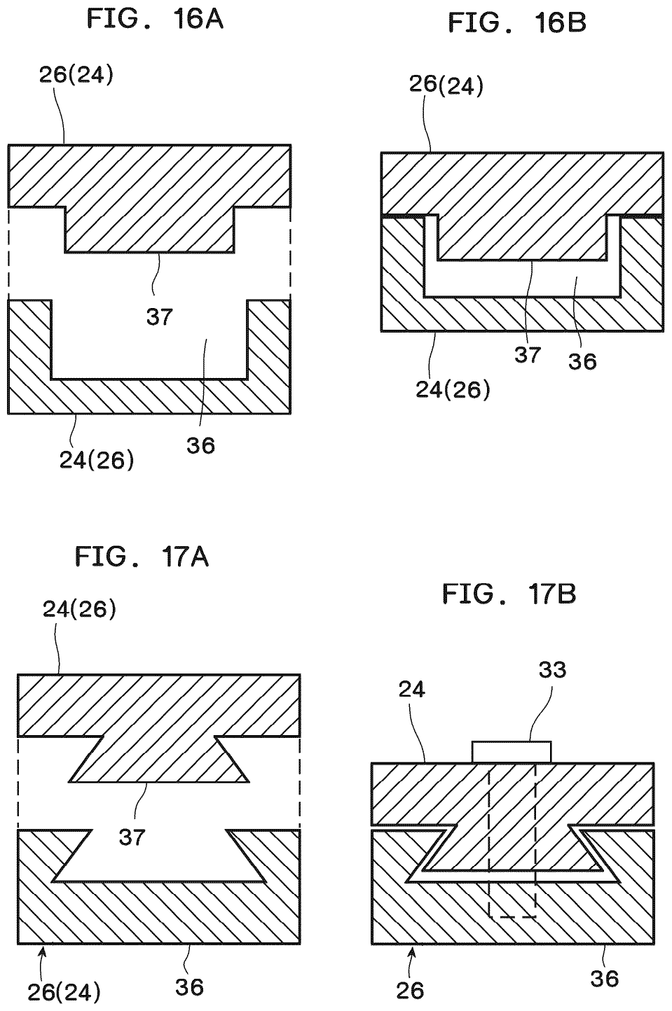

In this case, as illustrated in FIG. 17, when an opening 38 of the moving groove 36 is formed to be narrower in width than a groove bottom 39 and a cross-sectional shape of the moving body 37 is formed to a trapezoidal shape, the one-side handle 24 and the other-side handle 26 can only be slid without being detached, and the operation can be facilitated.

According to the present invention, there is provided a system configured to repair a fracture in a meniscus, the system including: a meniscus repairing device that includes a collecting member 1, a needle 16 and a handle 30, the collecting member 1 having an insertion space 6 therein and having an insertion body 8 inserted and arranged in the insertion space 6, entry of the insertion body 8 being allowed through the insertion space 6 of the collecting member 1, the needle 16 having an insertion space 17 therein and having a suture thread 5 arranged in the insertion space 17, entry of the suture thread 5 being allowed through the insertion space 17 of the needle 16, the needle 16 including a suture thread 5 having a certain length configured to be at least partly passed into a tissue, the suture thread 5 being attached in the needle 16, the suture thread 5 that has advanced from the needle 16 passing through the opening 3 of the collecting member 1, and being held by the insertion body 8 in a state of protruding to the outside of the collecting member 1, the held suture thread 5 being taken out from the body together with the collecting member 1, the handle 30 having an attachment tube 23 that allows the collecting member 1 to move in the length direction of the collecting member 1 to be fixed at a desired position, a one-side handle 24 to which the attachment tube 23 is attached, an attachment tube 25 that allows the needle 16 to move in the length direction of the needle 16 to be fixed at a desired position, and an other-side handle 26 to which the attachment tube 25 is attached; and a positioning instrument K configured to change attachment positions of the one-side handle 24 and the other-side handle 26, to allow the suture thread 5 that has advanced from the needle 16 to be toward the opening 3 of the collecting member 1.

Note that the needle 16 has any desired configuration, and as an example, the needle 16 is configured to form the insertion space 17 through which the suture thread 5 can be inserted inside by a tubular member made of metal.

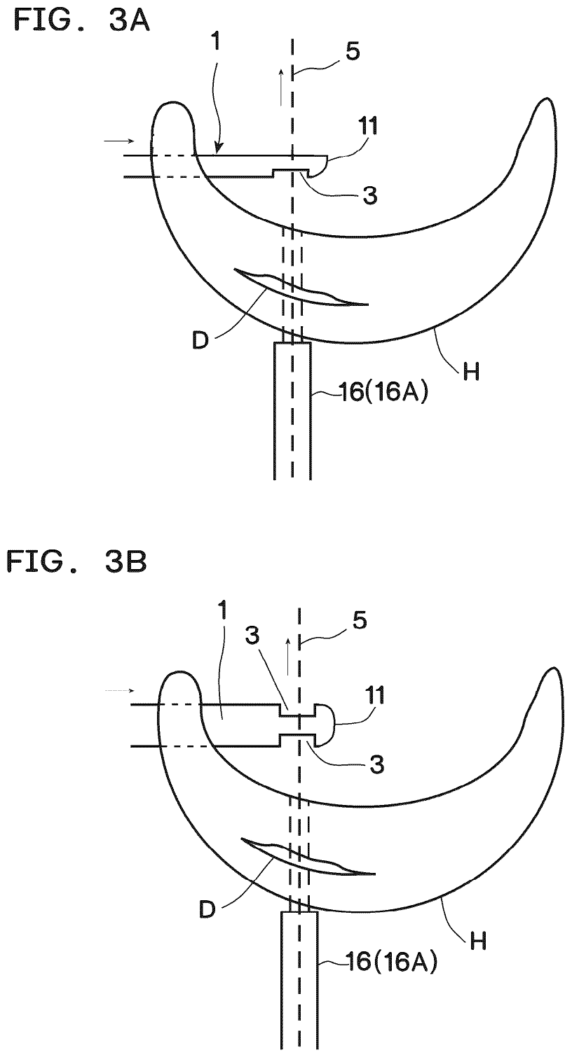

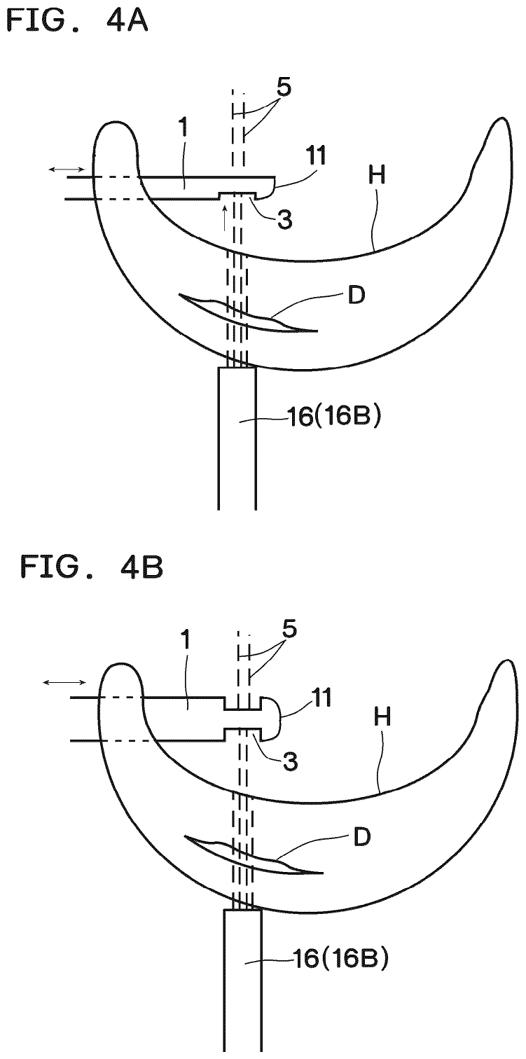

In addition, the needle 16 is formed of a single tubular member in an example of the present embodiment (FIG. 3 and the like), but may have a double tubular configuration, and may have a configuration in which the base side of an outer sub attachment tube 16A is fixed to the attachment tube 25, the needle 16 is inserted into the attachment tube 16A, and the suture thread 5 is inserted into the needle 16.

In the surgery of the meniscus H, there may be any desired method of using the needle 16, and the needle 16 may be configured to be directly inserted into and passed through the meniscus H, or the distal end of the needle 16 may be brought into contact with the meniscus H and the suture thread 5 may be inserted into an insertion hole (not illustrated) formed in the meniscus H.

Although not illustrated, the distal end of the sub attachment tube 16A may be brought into contact with the meniscus H, the hollow needle 16B may be directly inserted and passed through the meniscus H, and the suture thread 5 protruding from the hollow needle 16B may be fed and inserted through the opening 3 of the collecting member 1.

That is, the distal end of the sub attachment tube 16A is only brought into contact with the joint capsule, and is not stuck or inserted into the joint capsule or the meniscus tissue.

In addition, the sub attachment tube 16A may pierce the attachment tube 25, the distal end may be brought into contact with the outside of the joint capsule, the needle 16B and the suture thread 5 may be pierced to the sub attachment tube 16A, pierced into the joint capsule and the meniscus H, and may pass through the opening 3.

In other words, the sub attachment tube 16A is brought into contact with the joint capsule S, the needle (needle 16B) 16 passes through the joint capsule, the meniscus, and the opening 3 on the one of the pair of the collecting members 1, and if the needle (needle 16B) 16 passes through the opening 3 on the opposite side, the suture thread 5 also passes through the opening 3 on the opposite side. In this state, the needle (needle 16B) 16 is pulled out while leaving only the suture thread 5, and the needle (needle 16B) 16 loaded with the next suture thread 5 is inserted, and the above-described operation is repeated.

FIG. 19 is a schematic view for facilitating the configuration of the present invention, and illustrates a state in which the needle 16 (16A) is brought into contact with the joint capsule S.

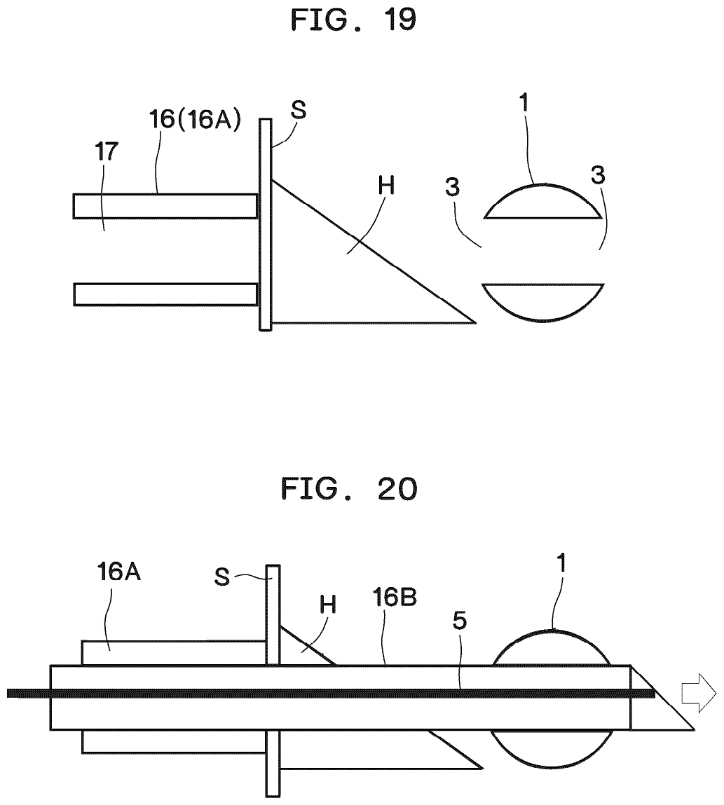

FIG. 20 is a schematic view of a state in which the needle 16 (16B) and the suture thread 5 are to pass through the opening 3 of the collecting member 1.

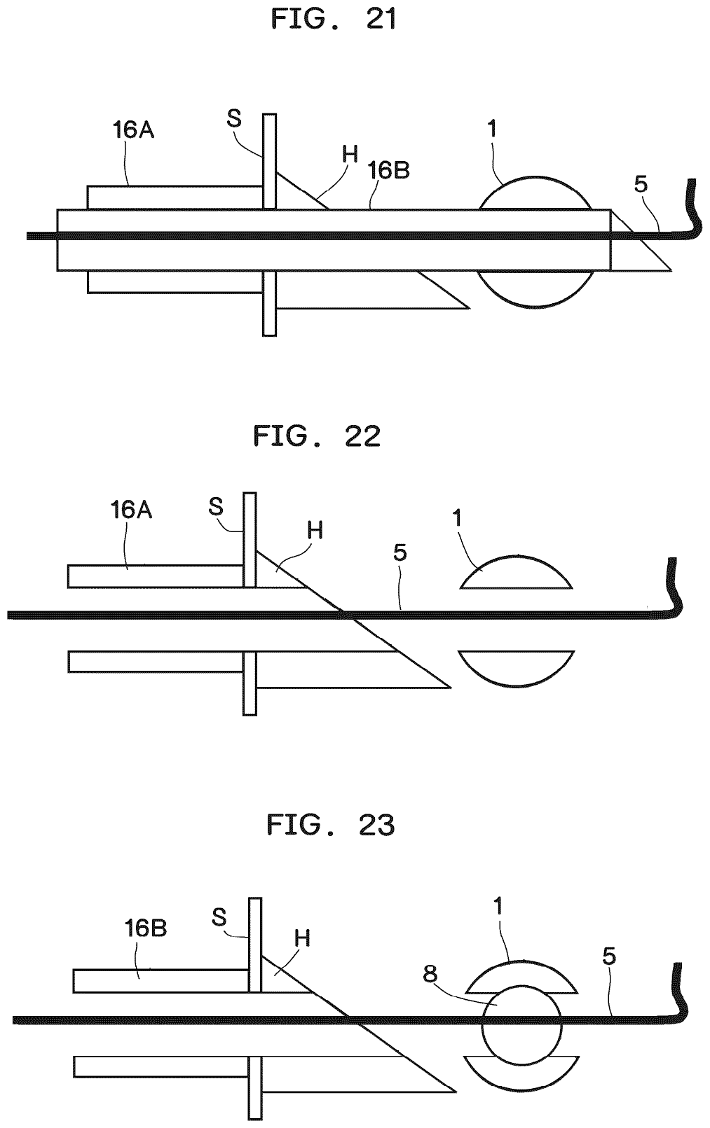

FIG. 21 is a schematic view of a state in which the needle 16 (16B) and the suture thread 5 have passed through the opening 3 of the collecting member 1.

FIG. 22 is a schematic view illustrating a state in which only the suture thread 5 is indwelled in the joint capsule S without the needle 16 (16B).

FIG. 23 is a schematic view of a state in which the insertion body 8 is inserted into the insertion space 6 of the collecting member 1 and the suture thread 5 is held.

Although the needle 16 (16B) is removed in FIGS. 22 and 23 for easy understanding, the needle 16 (16B) may be removed after the suture thread 5 is held by the insertion body 8.

### Operation of Embodiments

The present invention has the above-described configuration, and determines whether or not suturing of the meniscus H can be performed by inserting an arthroscope (endoscope) into a joint, confirming the damaged portion D recognized in the MRI inspection.

A skin of the joint capsule is incised according to the damaged portion on the rear side of the meniscus H to deploy the joint capsule, an optimal site of the joint capsule is incised, and the collecting member 1 is inserted into the joint from the incision site and installed in parallel to the damaged portion D as much as possible.

Next, the fixture 27 of the handle 30 is loosened, and in a state in which the one-side handle 24 and the other-side handle 26 can freely expand and contract, the distal end of the needle 16 and the opening 3 of the collecting member 1 are applied to the outside of the joint capsule so as to be orthogonal to each other within a possible range, and a temporary fixing state is obtained.

When a temporary fixing position is secured, the one-side handle 24 and the other-side handle 26 are fixed by the fixture 27, the sub attachment tube 16A inserted into the attachment tube 25 of the other-side handle 26 is further pressed against the joint capsule, the one-side handle 24 and the other-side handle 26 are fixed by the fixture 27, the suture thread 5 is pierced to the insertion space 17 of the needle 16B inserted into the sub attachment tube 16A, and the needle 16B is further pushed toward the meniscus H to pass through the opening 3 of the collecting member 1 as it is (FIG. 14).

In this state, the insertion body 8 is inserted from the base side insertion port 7 of the collecting member 1, and the distal end portion 10 of the insertion body 8 is brought into contact with the contact portion 11, whereby the distal end of the suture thread 5 is locked to the collecting member 1.

In this state, the needle 16 is removed to leave only the suture thread 5 in the joint capsule.

This operation is repeated to insert the second suture thread 5 into the opening 3 of the collecting member 1 to be locked therein.

When the two suture threads 5 can be fixed to the collecting member 1 by the insertion body 8, the collecting member 1 is pulled out of the joint, to take and collect the suture threads 5 out of the joint.

By connecting the distal ends of the two collected suture threads 5 outside the joint and pulling out the base of one of the suture threads 5, the other suture thread 5 comes out of the outside of the joint to the surfaces of the joint capsule, the meniscus, the damaged portion, and the meniscus, as a result, and turns back, and passes through the damaged portion, the meniscus, and the joint capsule in this order.

Next, both ends of the two suture threads 5 are ligated on the front side of the meniscus H to bring the damaged portion D into close contact with each other and promote repair.

In this case, if the collecting member 1 is curved as an option, the opening 3 of the collecting member 1 can be easily accessed to a pointed portion, which facilitates suturing and the operation of ligating.

As described above, even the two suture threads 5 passing through the meniscus H in the parallel state can be easily inserted into the opening 3 of the collecting member 1, the collecting member 1 being located on the rear side of the meniscus H in the intersecting direction with respect to the two suture threads 5, the insertion operation time can be extremely shortened, the operation of ligating in the joint capsule can be avoided, and the invasion to the patient can be reduced.

In addition, when the distal end of one of the two suture threads 5 is formed in a straight line shape and the distal end of the other suture thread 5 is formed in a loop shape, the one suture thread 5 of a straight line shape is pierced to the other suture thread 5 of a loop shape, the two suture threads 5 are connected together, and the connection operation of the two suture threads 5 is facilitated.

In this case, the distal end portion of the collecting member 1 of a straight line shape or a curved shape having a predetermined length of a semi-cylindrical shape or a spatula shape is provided with the opening 3 opened in the intersecting direction with respect to the length direction of the collecting member 1, the collecting member 1 can be easily inserted into the joint capsule having a narrow procedure space, and thus, when the suture thread 5 is inserted from the intersecting direction of the collecting member 1, the suture thread 5 can be inserted into the pair of the openings 3 of the collecting member 1 only by moving the meniscus H straight, and operations of inserting the suture thread 5 into the collecting member 1 and collecting the suture thread 5 from the collecting member 1 can be facilitated.

In addition, since the fitting groove 6A is provided on the inner surface of the collecting member 1, and the insertion body 8 having an axial rod shape is fitted and inserted into the fitting groove 6A from the base side of the collecting member 1, the suture thread 5 inserted into the pair of openings 3 of the collecting member 1 can be sandwiched between the outer surface of the insertion body 8 and the inner surface of the collecting member 1 and pulled out of the body, and the operation of ligating the suture thread 5 outside the body can be facilitated.

In addition, the distal end of the collecting member 1 raises the tip wall 9, and the opening 3 having a predetermined length in the length direction of the collecting member 1 is provided in the collecting member 1 on the base side relative to the tip wall 9. Therefore, the insertion body 8 fitted and inserted into the collecting member 1 can abut on the tip wall 9 of the collecting member 1 to sandwich the suture thread 5 in the collecting member 1 between the insertion body 8 and the tip wall 9, the operation of pulling out the suture thread 5 to the outside of the body can be facilitated, and the operation of ligating the suture thread 5 outside the body can be facilitated.

In addition, since the tip wall 9 of the collecting member 1 is formed on the contact portion 11 on which the distal end portion 10 of the insertion body 8 inserted from the base side of the collecting member 1 into the fitting groove 6A abuts, the distal end portion 10 of the insertion body 8 inserted into the collecting member 1 can abut on the contact portion 11 of the collecting member 1, to allow the distal end portion 10 of the insertion body 8 and the contact portion 11 of the collecting member 1 to lock the suture thread 5 in the collecting member 1, and the operation of pulling out the suture thread 5 to the outside of the body can be facilitated, and the operation of ligating the suture thread 5 outside the body can be facilitated.

In addition, since the distal end portion 10 of the insertion body 8 and the contact portion 11 of the collecting member 1 are configured such that an outer surface of the distal end portion 10 abuts on an inner surface of the contact portion 11 to relatively prevent the suture thread 5 from coming off, the distal end portion 10 of the insertion body 8 fitted and inserted into the collecting member 1 can abut on the contact portion 11 of the collecting member 1, to allow the distal end portion 10 of the insertion body 8 and the inner surface of the contact portion 11 to sandwich the suture thread 5, and the operation of pulling out the suture thread 5 to the outside of the body can be facilitated, and the operation of ligating the suture thread 5 outside the body can be facilitated.

In addition, at least the distal end portion 10 of the insertion body 8 is formed to have flexibility, and the insertion body 8 is configured to prevent cutting of the suture thread 5 when the distal end portion 10 of the insertion body 8 passes above or near the opening 3 of the collecting member 1. Therefore, the operation of pulling out the suture thread 5 to the outside of the body can be facilitated while preventing the cutting of the suture thread 5, and the operation of ligating the suture thread 5 outside the body can be facilitated.

In addition, since the opening 3 of the collecting member 1 is formed to have a size that allows at least the suture thread 5 and/or the needle 16 to be inserted to the opening 3, the suture thread 5 can be pierced to the opening 3 of the collecting member 1, and the insertion of the suture thread 5 into the collecting member 1 is facilitated.

In another embodiment of FIG. 1B, the distal end portion of the collecting member 1 of a straight line shape having a predetermined length of a hollow tubular shape is provided with the opening 3 opened in the intersecting direction with respect to the axial direction of the collecting member 1, and thus when the collecting member 1 is inserted in a state of being parallel to the affected part (damaged portion D) of the meniscus H and the suture thread 5 is inserted from the intersecting direction of the collecting member 1 in this state, the suture thread 5 can be inserted into the pair of the openings 3 of the collecting member 1 only by moving the meniscus H straight, and the operation of inserting the suture thread 5 into the collecting member 1 can be facilitated.

Since the collecting member 1 is provided with the insertion space 6 therein, and is configured such that the insertion body 8 having an axial rod shape is inserted from the base side insertion port 7 provided on the base side of the collecting member 1, the suture thread 5 inserted into the pair of openings 3 of the collecting member 1 can be sandwiched between the outer surface of the insertion body 8 and the inner surface of the collecting member 1 and pulled out of the body, and the connection operation of the suture thread 5 outside the body can be facilitated.

The distal end of the collecting member 1 is closed by the tip wall 9, and the pair of openings 3 each having a predetermined length in the length direction of the collecting member 1 are provided in the collecting member 1 on the base side relative to the tip wall 9 so as to face each other in the intersecting direction with respect to the length direction of the collecting member 1. Therefore, the insertion body 8 inserted into the collecting member 1 can abut on the tip wall 9 of the collecting member 1 to allow the suture thread 5 in the collecting member 1 to be interposed between the insertion body 8 and the tip wall 9, the operation of pulling out the suture thread 5 to the outside of the body can be facilitated, and the operation of ligating the suture thread 5 outside the body can be facilitated.

In addition, in another embodiment of FIG. 1C, since the distal end side of the collecting member 1 beyond the opening 3 is formed in the fitting portion 11A to which the distal end portion 10 of the insertion body 8 inserted into the insertion space 6 is fitted, the distal end portion 10 of the insertion body 8 inserted into the collecting member 1 is fitted to the fitting portion 11A of the distal end of the collecting member 1, to allow the suture thread 5 in the collecting member 1 to be interposed between both the upper and lower surfaces of the fitting portion 11A and the inner surface of the tip wall 9, and the operation of pulling out the suture thread 5 to the outside of the body can be facilitated, and the operation of ligating the suture thread 5 outside the body can be facilitated.

In addition, since the distal end portion 10 of the insertion body 8 and the contact portion (fitting portion 11A) 11 of the collecting member 1 are configured such that an outer surface of the distal end portion 10 is closely fitted to the inner surface of the contact portion 11 to relatively prevent the suture thread 5 positioned in the contact portion (fitting portion 11A) 11 from coming off, the distal end portion 10 of the insertion body 8 inserted into the collecting member 1 can abut (be fitted to) on the contact portion 11 of the collecting member 1, to allow the suture thread 5 in the collecting member 1 to be interposed between the distal end portion 10 and the contact portion 11, and the operation of pulling out the suture thread 5 to the outside of the body can be facilitated, and the operation of ligating the suture thread 5 outside the body can be facilitated.

Since the portion close to the base side relative to the opening 3 of the collecting member 1 serves as the insertion portion 15 into which the insertion body 8 is inserted, and the inner diameter of the insertion portion 15 is formed to be larger than the outer diameter of the insertion body 8, the insertion body 8 can be easily and smoothly inserted into the collecting member 1, fitting of the distal end portion 10 of the insertion body 8 to the distal end portion 10 of the collecting member 1 can be facilitated, the operation of pulling out the suture thread 5 to the outside of the body can be facilitated, and the operation of ligating the suture thread 5 outside the body can be easily performed.

The opening 3 of the collecting member 1 is formed to have a size that allows at least the needle 16 and the suture thread 5 to pass therethrough. However, since the opening 3 is formed to have a size that allows the needle 16 attached with the suture thread 5 passing through the meniscus H to pass therethrough, the suture thread 5 can pass through the opening 3 of the collecting member 1 together with the needle 16, and insertion of the suture thread 5 into the collecting member 1 is facilitated.

The distal end of the collecting member 1 of a straight line shape having a predetermined length, the collecting member 1 being hollow and provided with the insertion space 6 inside, is closed by the tip wall 9, the pair of the openings 3 each having a predetermined length in the length direction of the collecting member 1 are provided in the collecting member 1 on the base side of the collecting member 1 for the predetermined length from the tip wall 9 so as to face each other in the intersecting direction with respect to the length direction of the collecting member 1, the opening 3 is opened to have a size that allows the suture thread 5 or the needle 16 to be inserted thereinto, the base side insertion port 7 for inserting the insertion body 8 is formed on the base side of the collecting member 1, and a distal end side of the collecting member 1 beyond the opening 3 is formed in the contact portion 11 on which the distal end portion 10 of the insertion body 8 inserted into the insertion space 6 abuts. Therefore, the contact portion 11 of the collecting member 1 is inserted from the outside of the body into the approach path formed on the rear side of the meniscus H, the opening 3 of the collecting member 1 is positioned on the back side of the damaged portion (fractured portion) D of the meniscus H, whereas, the sub attachment tube 16A is brought into contact with the joint capsule so as to pass through the damaged portion D of the meniscus H from the approach path of the front side of the meniscus H, the needle (needle 16B) 16 and the suture thread 5 are allowed to enter the and the joint capsule, one or both of the needle 16 and the suture thread 5 are passed through the opening 3 of the collecting member 1, similarly, another suture thread 5 is passed through the opening 3, the insertion body 8 is inserted into the insertion space 6 of the collecting member 1 in a state of leaving the distal ends of the two suture threads 5 outside the collecting member 1, the distal end portion 10 of the insertion body 8 is closely fitted to the contact portion 11 of the collecting member 1, the collecting member 1 together with the distal ends of the two suture threads 5 are pulled out to the outside of the body, and the two suture threads 5 that have been taken out are connected together, and the base of the suture thread 5 having a loop shape is pulled to suture the damaged portion D of the meniscus H with the two suture threads 5.

The collecting member 1 is configured to be insertable into the casing 20 having a larger diameter than the outer diameter of the collecting member 1 inserted into the back side of the meniscus H and having a straight line hollow tubular shape. Therefore, the casing 20 is inserted in a state of being parallel to the affected part (damaged portion (fractured portion) D) of the meniscus H, the collecting member 1 is inserted into the casing 20, the opening 3 of the collecting member 1 is made to protrude to the distal end side beyond the distal end side of the casing 20, the suture thread 5 or the needle 16 is inserted into the opening 3 of the collecting member 1, the distal end of the suture thread 5 is locked to the distal end of the collecting member 1, the collecting member 1 is taken out of the body while the casing 20 is indwelled in the body in this state, the suture thread 5 is pulled out of the body, and the operation of ligating the suture thread 5 can be performed outside the body.

When the collecting member 1 is inserted into the casing 20 again, the opening 3 of the collecting member 1 is positioned in the vicinity of the meniscus H, the suture thread 5 is locked to the collecting member 1, and the suture operation of the damaged portion D of the meniscus H can be easily repeated.

The insertion body 8 is formed of a material having flexibility and rigidity such as vinyl and silicon such that the distal end of the insertion body 8 reaches the distal end portion 10 of the collecting member 1 when inserted into the insertion space 6 of the collecting member 1. Therefore, insertion of the insertion body 8 can be easily performed. Since the opening 3 of the collecting member 1 is formed in a rectangular slit shape having a width of 1 to 2 mm and a length of 12 to 20 mm, the suture thread 5 can be easily inserted from the intersecting direction.

Since the handle 30 includes the one-side handle 24 provided with the attachment tube 23 to which the collecting member 1 having the opening 3 at the distal end and allowing the suture thread 5 to be inserted is attached, and the other-side handle 26 provided with the attachment tube 25 to which the sub attachment tube 16A for inserting and guiding the suture thread 5 in the vicinity of the damaged portion D of the meniscus H is attached, the one-side handle 24 and the other-side handle 26 being fixed with the fixture 27, the operation of inserting the insertion body 8 into the collecting member 1 is enabled by holding the handle 30, and the operation of inserting the insertion body 8 is facilitated.

The attachment tube 23 and the attachment tube 25 are arranged to each other so as to position the distal end of the needle 16 so as to intersect the opening 3 in a state where the opening 3 of the collecting member 1 is positioned at a predetermined position in the vicinity of the meniscus H. Therefore, the collecting member 1 attached to the handle 30 and the needle 16 are positioned so as to cross each other in the body, and insertion work of the collecting member 1 and the needle 16 is facilitated. That T is, the handle 30 includes the one-side handle 24 having the attachment tube 23 and the other-side handle 26 movably attached to the one-side handle 24, in which the base of the one-side handle 24 and the base of the other-side handle 26 are overlapped and fixed by a fastener 35. Therefore, the length of the handle 30 can be switched and adjusted, and operability and workability can be improved.

The attachment tube 23 and the attachment tube 25 of the handle 30 are arranged to each other to position the distal end of the needle 16 so as to intersect the opening 3 in a state where the opening 3 of the collecting member 1 is positioned at a predetermined position in the vicinity of the meniscus H. Therefore, the predicted position at which the suture thread 5 will be inserted into the damaged portion D of the meniscus H is set in advance, the orientation of the attachment tube 23 of the handle 30 is set such that the opening 3 of the collecting member 1 is positioned at the set position, and then the one-side handle 24 and the other-side handle 26 are combined and fixed such that the attachment tube 25 of the other-side handle 26 is positioned at an angle at which the distal end of the needle 16 will be inserted into a position at which the opening 3 of the collecting member 1 will be positioned.

Therefore, it is possible to simulate the arrangement relationship among the damaged portion D of the meniscus H, the opening 3 of the collecting member 1, and the distal end position of the needle 16 in advance outside the body. Based on this simulation, it is only necessary to cause the collecting member 1 and the needle 16 to contact the joint capsule, cause the collecting member 1 to be inserted into the joint capsule, and cause the suture thread 5 to be inserted from the needle 16 into the opening 3 of the collecting member 1 and locked thereto, and thus the suture operation can be performed in a very short time. A system configured to repair a fracture in a meniscus, the system including: a meniscus repairing device that includes a collecting member 1, a needle 16 and a handle 30, the collecting member 1 having an insertion space 6 therein and having an insertion body 8 inserted and arranged in the insertion space 6, entry of the insertion body 8 being allowed through the insertion space 6 of the collecting member 1, the needle 16 including a suture thread 5 having a certain length configured to be at least partly passed into a tissue, the suture thread 5 being attached in the needle 16, the suture thread 5 that has advanced from the needle 16 passing through the opening 3 of the collecting member 1, and being held by the insertion body 8 in a state of protruding to the outside of the collecting member 1, the held suture thread 5 being taken out from the body together with the collecting member 1, the handle 30 having an attachment tube 23 that allows the collecting member 1 to move in the length direction of the collecting member 1 to be fixed at a desired position, a one-side handle 24 to which the attachment tube 23 is attached, an attachment tube 25 that allows the needle 16 to move in the length direction of the needle 16 to be fixed at a desired position, and an other-side handle 26 to which the attachment tube 25 is attached; and a positioning instrument K configured to change attachment positions of the one-side handle 24 and the other-side handle 26, to allow the suture thread 5 that has advanced from the needle 16 to be toward the opening 3 of the collecting member 1, and suturing of the meniscus H is facilitated.

That is, first, a position of the damaged portion D of the meniscus H in the knee is detected by MRI inspection. On the basis of this result, an insertion depth of the one-side handle 24 of the handle 30 of the collecting member 1 into the attachment tube 23 is determined.

Next, the insertion depth of the needle 16 into the attachment tube 23 of the other-side handle 26 of the handle 30 is determined such that the distal end of the suture thread 5 is positioned in a straight line manner with respect to the opening 3 of the collecting member 1 attached to the attachment tube 25 of the handle 30. In this state, the orientation of the attachment tube 23 of the one-side handle 24 of the handle 30 and the orientation of the attachment tube 25 of the other-side handle 26 are determined, and the collecting member 1, the handle 30, and the needle 16 are arranged so as to be inserted into the opening 3 of the collecting member 1 when the distal ends of the needle 16 and the suture thread 5 are stretched as they are.

With this configuration, it is possible to easily simulate the approach of the handle 30, the collecting member 1, and the needle 16 of the suturing instrument to the site of the damaged portion D of the meniscus H in the joint capsule at the outside of the body.

While the foregoing description and drawings represent the preferred embodiment of the present invention, it will be understood that various additions, modifications, combinations and/or substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed embodiments are, therefore, to be considered in all respects as illustrative and not restrictive, the scope of the invention being indicated by the appended claims and not limited to the foregoing description.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention, as defined by the appended claims.

What is claimed is:

1. A suturing instrument in which a distal end portion of a collecting member 1 of a straight line shape or a curved shape having a predetermined length of a semi-cylindrical shape or a spatula shape is provided with an opening 3 opened in the intersecting direction with respect to the length direction of the collecting member 1.

2. The suturing instrument according to claim 1, wherein the collecting member 1 has a configuration in which a fitting groove 6A is provided on an inner surface of the collecting member 1, and an insertion body 8 of an axial rod shape is fitted and inserted into the fitting groove 6A from a base side of the collecting member 1.

3. The suturing instrument according to claim 2, wherein a tip wall 9 is provided at the distal end of the collecting member 1, and an opening 3 having a predetermined length in the length direction of the collecting member 1 is provided on the collecting member 1 on the base side relative to the tip wall 9.

4. The suturing instrument according to claim 3, wherein the tip wall 9 of the collecting member 1 is formed to be a contact portion 11 on which the distal end portion 10 of the insertion body 8 inserted into the fitting groove 6A from the base side of the collecting member 1 abuts.

5. The suturing instrument according to claim 2, wherein the opening 3 of the collecting member 1 is formed to have a size that allows at least the suture thread 5 and/or a needle 16 to be inserted into the opening 3.

6. A suturing instrument in which a distal end portion of a collecting member 1 of a straight line shape or a curved shape having a predetermined length of a hollow tubular shape is provided with an opening 3 opened in the intersecting direction with respect to the axial direction of the collecting member 1.

7. The suturing instrument according to claim 6, wherein the distal end of the collecting member 1 is closed with the tip wall 9, the openings 3 each having a predetermined length in the length direction of the collecting member 1 are provided on the base side of the collecting member 1 relative to the tip wall 9 for the predetermined length from the tip wall 9 so as to face each other in the intersecting direction with respect to the length direction of the collecting member 1.

8. The suturing instrument according to claim 6, wherein a portion close to the base side relative to the opening 3 of the collecting member 1 is an insertion portion 15 into which the insertion body 8 is inserted, and an inner diameter of the insertion portion 15 is formed to be larger than an outer diameter of the insertion body 8.

9. The suturing instrument according to claim 6, wherein the opening 3 of the collecting member 1 is formed to have a size that allows at least the suture thread 5 and/or a needle 16 to be inserted into the opening 3.

10. The suturing instrument according to claim 8, wherein the insertion body 8 is formed of a material having flexibility and rigidity such as vinyl and silicon such that the distal end portion 10 of the insertion body 8 reaches the distal end of the collecting member 1 when inserted into the insertion space 6 of the collecting member 1.

11. A suturing positioning instrument according to claim 10 comprising a handle 30, the handle including: one-side handle 24 provided with an attachment tube 23 to which the collecting member 1 having the opening 3 at the distal end and allowing the suture thread 5 to be inserted is attached; and other-side handle 26 provided with an attachment tube 25 to which the needle 16 for inserting and guiding the suture thread 5 in the vicinity of the damaged portion D of the meniscus H is attached, wherein the one-side handle 24 and the other-side handle 26 overlap each other and are fixed together by a fixture 27 such that an overlapped length is adjustable.

12. The suturing positioning instrument according to claim 11, wherein the one-side handle 24 and the other-side handle 26 are configured to fix the collecting member 1 and the needle 16 in a state of intersecting each other at any desired angle by adjusting an overlapping length between the one-side handle 24 and the other-side handle 26.

13. The suturing positioning instrument according to claim 10, wherein the attachment tube 23 and the attachment tube 25 are arranged to each other such that the distal end of the needle 16X (needle 16?) is positioned to intersect the opening 3 in a state where the opening 3 of the collecting member 1 is positioned at a predetermined position in the vicinity of the meniscus H.

14. A system configured to repair a fracture in a meniscus, the system including: a meniscus repairing device that includes a collecting member 1, a needle 16 and a handle 30, the collecting member 1 having an insertion space 6 therein and having an insertion body 8 inserted and arranged in the insertion space 6, entry of the insertion body 8 being allowed through the insertion space 6 of the collecting member 1, the needle 16 having an insertion space 17 therein and having a suture thread 5 arranged in the insertion space 17, entry of the suture thread 5 being allowed through the insertion space 17 of the needle 16, the needle 16 including a suture thread 5 having a certain length configured to be at least partly passed into a tissue, the suture thread 5 being attached in the needle 16, the suture thread 5 that has advanced from the needle 16 passing through the opening 3 of the collecting member 1, and being held by the insertion body 8 in a state of protruding to the outside of the collecting member 1, the held suture thread 5 being taken out from the body together with the collecting member 1, the handle 30 having an attachment tube 23 that allows the collecting member 1 to move in the length direction of the collecting member 1 to be fixed at a desired position, a one-side handle 24 to which the attachment tube 23 is attached, an attachment tube 25 that allows the needle 16 to move in the length direction of the needle 16 to be fixed at a desired position, and an other-side handle 26 to which the attachment tube 25 is attached; and a positioning instrument K configured to change attachment positions of the one-side handle 24 and the other-side handle 26, to allow the suture thread 5 that has advanced from the needle 16 to be toward the opening 3 of the collecting member 1.
